(19) United States
(12) Patent Application Publication
(10) Pub. No.: US 2003/0219479 A1
(43) Pub. Date: Nov. 27, 2003

(54) Multi-layer mucoadhesive drug delivery device with bursting release layer

(75) Inventors: Li-Lan H. Chen, Edison, NJ (US); Alfred Liang, Edison, NJ (US)

Correspondence Address: ALLEN BLOOM, C/O DECHERT, PRINCETON PIKE CORPORATION CENTER, P.O. BOX 5218, PRINCETON, NJ, 08543-5218, US

(73) Assignee: Lavipharm Laboratories Inc.

(21) Appl. No.: 10/409,681

(22) Filed: Apr. 8, 2003

Related U.S. Application Data
(60) Provisional application No. 60/370,834, filed on Apr. 8, 2002.

(51) Int. Cl. A61K 9/46

(52) U.S. Cl. 424/466

(57) ABSTRACT

Drug delivery devices for administering an active agent or a combination of active agents to a subject are provided. Specifically, multi-layer mucoadhesive drug delivery device are provided, including: (a) a mucoadhesive layer, containing at least one non-ionic polymer, at least one anionic polymer, at least one swelling modifier, and at least one buffering agent; (b) an effervescent layer, containing at least one permeation enhancer, an effervescent couple, comprising an anhydrous acid and an alkalizing agent, and at least one binder; and, (c) at least one active agent; wherein the at least one active agent is contained in both the mucoadhesive layer and the effervescent layer; wherein the at least one active agent contained in the effervescent layer is released from the drug delivery device along with the at least one permeation enhancer within 10 minutes, more preferably within 5 minutes, most preferably within 1 minute, of administration of the drug delivery device to a subject and wherein the at least one active agent contained in the mucoadhesive layer is released from the drug delivery device over a period of at least 8 hours, more preferably at least 12 hours, most preferably at least 24 hours, preferably with near zero-order kinetics, more preferably with zero-order kinetics. Methods of administering active agents to a subject using such multi-layer mucoadhesive drug deliver devices are also provided.

The present invention relates to multi-layer mucoadhesive drug delivery devices having a bursting release layer for administering an active agent or combination of active agents to a subject. Specifically, the present invention relates to a multi-layer mucoadhesive drug delivery device, including: (a) a mucoadhesive layer, containing at least one non-ionic polymer, at least one anionic polymer, at least one swelling modifier, and at least one buffering agent; (b) an effervescent layer, containing at least one permeation enhancer, an effervescent couple, comprising an anhydrous acid and an alkalizing agent, and at least one binder; and, (c) at least one active agent; wherein the at least one active agent is contained in both the mucoadhesive layer and the effervescent layer; wherein the at least one active agent contained in the effervescent layer is released from the drug delivery device along with the at least one permeation enhancer within 10 minutes, more preferably within 5 minutes, most preferably within 1 minute, of administration of the drug delivery device to a subject and wherein the at least one active agent contained in the mucoadhesive layer is released from the drug delivery device over a period of at least 8 hours, more preferably at least 12 hours, most preferably at least 24 hours, preferably with near zero-order kinetics, more preferably with zero-order kinetics. The present invention also relates to methods for using such multi-layer mucoadhesive drug deliver devices to provide a sustained, controlled release of an active agent or combination of active agents to a subject, preferably a near zero-order release of the active agent or combination of active agents, more preferably a zero-order release of the active agent or combination of active agents.

Prescription and over-the-counter medications and other pharmaceutical products have traditionally been administered through oral ingestion, nasal sprays, injections and suppositories. For example, many pharmaceutical dosage forms are administrated orally in the form of solid shaped articles such as tablets, pills, caplets and capsules that retain their shape under moderate pressure. Generally these dosage forms are designed to be swallowed whole or chewed to deliver the medication with adequate amounts of liquid. Some patients, particularly pediatric and geriatric patients, have difficulty swallowing or chewing such solid dosage forms. Certain patients such as children or animals often resist taking medications, and may try to hide such dosage forms in order to spit it out later. In addition, many pediatric and geriatric patients are unwilling to take such solid dosage forms because they have difficulty swallowing them even when liquids are consumed therewith. Furthermore, the availability of liquids at the time of administering medications may be limited for certain patients and may be restricted for certain diseases and/or treatments.

Chewable tablets provide some advantages over conventional tablets. Such chewable tablets, however, are not suitable for children wearing braces and the taste of certain active agents may be unpleasant and difficult to mask in a chewable tablet. In addition, the use of chewable tablets may not eliminate the desire or need to administer water or some other liquid therewith.

Furthermore, the standard oral dosage forms, such as tablets, pills, caplets, and capsules, are designed for short residence time in the mouth. Absorption of the active agent from these dosage forms typically occurs in the gastrointestinal (GI) tract, after the active agent has separated from the dosage form and dissolved in the gastric fluids. For some active agents, it is desirable to achieve absorption through a mucosal tissue in order to accelerate onset of the therapeutic effect.

Many active agents are poorly absorbed, even after they are dispersed in the stomach, because of low solubility or slow dissolution rate in the gastric fluids. Tablets may be formulated so as to be quick dissolving. These tablets are commonly placed on the tongue and disintegrate rapidly in the oral cavity. These dosage forms, however, are not fixed to a mucosal tissue and may move around in the mouth. Consequently, these dosage forms do not overcome the risk associated with choking or gagging that occurs with subjects having limited control of their swallowing reflexes.

### GLOSSARY

The following definitions are provided to facilitate an understanding of certain terms used frequently herein.

The term “controlled release” as used herein and in the appended claims means that a predetermined dosage of an active agent or combination of active agents is administered to a subject over a period of time.

The term “mucosal tissue” as used herein and in the appended claims means any moist mucosal surface of the subjects body as deemed appropriate for the systemic or local delivery of an active agent or combination of active agents including oral, nasal, vaginal, rectal and ocular tissues. The term “oral tissue(s)” as used herein and in the appended claims includes lingual, sub-lingual, buccal, gingival and palatal surfaces; most preferably lingual, sub-lingual and buccal surfaces.

The term “permeation enhancer” as used herein and in the appended claims means a natural or synthetic molecule which facilitates the absorption of a given active agent or combination of active agents through a mucosal tissue.

The term “release period” as used herein and in the appended claims means the period of time subsequent to administration of a drug delivery devices of the present invention during which the delivery device releases an active agent or combination of active agents to a subject.

The term “subject” as used herein and in the appended claims means an animal, preferably a mammal, more preferably canines and primates, most preferably humans.

The term “sustained release” as used herein and in the appended claims means the continual release of an active agent or combination of active agents over a period of time.

The term “zero order release” in relation to release kinetics as used herein and in the appended claims means that the rate of release of the active agent or active agents from a delivery device is a linear function with time.

### SUMMARY OF THE INVENTION

In a preferred embodiment of the present invention, drug delivery devices are provided which contain: (a) a mucoadhesive layer, containing: (i) at least one nonionic polymer, (ii) at least one anionic polymer, (iii) at least one swelling modifier, and (iv) at least one buffering agent; (b) an effervescent layer, containing: (i) at least one permeation enhancer, (ii) at least one effervescent couple, each such effervescent couple containing an anhydrous acid and an alkalizing agent, and (iii) at least one binder; and, (c) at least one active agent.

In a preferred aspect of this embodiment, the at least one nonionic polymer preferably constitutes 10 to 60 wt % of the mucoadhesive layer.

In another preferred aspect of this embodiment, the at least one anionic polymer preferably constitutes 10 to 60 wt % of the mucoadhesive layer.

In another preferred aspect of this embodiment, the at least one swelling modifier preferably constitutes 0.1 to 50 wt % of the mucoadhesive layer.

In another preferred aspect of this embodiment, the at least one permeation enhancer preferably constitutes 0.01 to 20 wt % of the effervescent layer.

In another preferred aspect of this embodiment, the at least one effervescent couple preferably constitutes 50 to 95 wt % of the effervescent layer.

In another preferred aspect of this embodiment, the at least one active agent may preferably be contained in both the mucoadhesive layer and the effervescent layer. Preferably, the at least one active agent preferably constitutes 0.01 to 50 wt % of the drug delivery device.

In another preferred aspect of this embodiment, the at least one active agent contained in the effervescent layer preferably is released from the drug delivery device along with the at least one permeation enhancer within 10 minutes, more preferably within five minutes, most preferably within 1 minute of administration of the drug delivery device to a subject.

In another preferred aspect of this embodiment, the at least one active agent contained in the mucoadhesive layer preferably is released from the drug delivery device over a period of at least 8 hours, more preferably at least 12 hours, most preferably at least 24 hours, with near zero-order kinetics after administration of the drug delivery device to a subject, more preferably with zero order kinetics.

In another preferred aspect of this embodiment, the effervescent layer preferably is nonmucoadhesive.

In another preferred aspect of this embodiment, the drug delivery devices of the present invention preferably completely erodes from the site of application.

In another preferred aspect of this embodiment, the at least one swelling modifier may preferably include swelling retarders and/or swelling promoters.

In another preferred aspect of this embodiment, the drug delivery devices of the present invention may optionally contain additional ingredients including taste modifiers, coloring agents, glidants and lubricants. When included, such optional additional ingredients may preferably constitute 0.01 to 10 wt % of the drug delivery device.

In another preferred embodiment of the present invention, drug delivery devices are provided which contain: (a) a mucoadhesive layer, containing: (i) at least one nonionic polymer, (ii) at least one anionic polymer, (iii) at least one swelling modifier, and (iv) at least one buffering agent; (b) an effervescent layer, containing: (i) at least one permeation enhancer, (ii) at least one effervescent couple, each such effervescent couple containing an anhydrous acid and an alkalizing agent, and (iii) at least one binder; (c) at least one active agent; and, (d) at least one glidant.

In a preferred aspect of this embodiment, the at least one nonionic polymer preferably constitutes 10 to 60 wt % of the mucoadhesive layer.

In another preferred aspect of this embodiment, the at least one anionic polymer preferably constitutes 10 to 60 wt % of the mucoadhesive layer.

In another preferred aspect of this embodiment, the at least one swelling modifier preferably constitutes 0.1 to 50 wt % of the mucoadhesive layer.

In another preferred aspect of this embodiment, the at least one permeation enhancer preferably constitutes 0.01 to 20 wt % of the effervescent layer.

In another preferred aspect of this embodiment, the at least one effervescent couple preferably constitutes 50 to 95 wt % of the effervescent layer.

In another preferred aspect of this embodiment, the at least one active agent may preferably be contained in both the mucoadhesive layer and the effervescent layer. Preferably, the at least one active agent preferably constitutes 0.01 to 50 wt % of the drug delivery device.

In another preferred aspect of this embodiment, the at least one active agent contained in the effervescent layer preferably is released from the drug delivery device along with the at least one permeation enhancer within 10 minutes, more preferably within five minutes, most preferably within 1 minute, of administration of the drug delivery device to a subject.

In another preferred aspect of this embodiment, the at least one active agent contained in the mucoadhesive layer preferably is released from the drug delivery device over a period of at least 8 hours, more preferably at least 12 hours, most preferably at least 24 hours, with near zero-order kinetics, most preferably with zero-order kinetics, after administration of the drug delivery device to a subject.

In another preferred aspect of this embodiment, the effervescent layer preferably is nonmucoadhesive.

In another preferred aspect of this embodiment, the drug delivery device completely erodes from the site of application.

In another preferred aspect of this embodiment, the at least one swelling modifier may include swelling retarders and swelling promoters.

In another preferred aspect of this embodiment, the drug delivery device may optionally contain additional ingredients including taste modifiers, coloring agents, glidants and lubricants. When included, such optional additional ingredients may preferably constitute 0.01 to 10 wt % of the drug delivery device.

In another preferred embodiment of the present invention, drug delivery devices are provided which contain: (a) a mucoadhesive layer, containing: (i) at least one nonionic polymer, (ii) at least one anionic polymer, (iii) at least one swelling modifier, and (iv) at least one buffering agent; (b) an effervescent layer, containing: (i) at least one permeation enhancer, (ii) at least one effervescent couple, each such effervescent couple containing an anhydrous acid and an alkalizing agent, and (iii) at least one binder; (c) at least one active agent; and, (d) at least one glidant; wherein the at least one active agent is contained in both the mucoadhesive layer and the effervescent layer; wherein the at least one glidant is present in both the mucoadhesive layer and the effervescent layer; wherein the mass ratio of anionic to non-ionic polymers in the mucoadhesive layer is 1:6 to 6:1; wherein the mass ratio of the (I) swelling modifier(s) to the (II) combined mass of the anionic polymer(s) and the nonionic polymer(s) is 1:50 to 1:1; wherein the drug delivery device preferably completely erodes from the site of application on the mucosal tissue; wherein the at least one active agent contained in the effervescent layer is released from the drug delivery device along with the at least one permeation enhancer within ten minutes, more preferably within five minutes, most preferably within one minute, of administration of the drug delivery device to a subject through application of the drug delivery device to a mucosal tissue selected from an oral mucosal tissue and a vaginal mucosal tissue and wherein the at least one active agent contained in the mucoadhesive layer is released from the drug delivery device over a period of at least 8 hours, more preferably at least 12 hours, most preferably at least 24 hours, with near zero-order kinetics, more preferably with zero-order kinetics.

In another preferred embodiment of the present invention, a method for delivering an active agent to a subject is provided, wherein a drug delivery device is administered to a mucosal tissue of the subject; wherein the delivery device contains: (a) a mucoadhesive layer, comprising: (i) at least one non-ionic polymer, (ii) at least one anionic polymer, (iii) at least one swelling modifier, and (iv) at least one buffering agent; (b) an effervescent layer, containing: (i) at least one permeation enhancer, (ii) at least one effervescent couple, each such effervescent couple containing an anhydrous acid and an alkalizing agent, and (iii) at least one binder; and, (c) at least one active agent; wherein the at least one active agent is contained in both the mucoadhesive layer and the effervescent layer; wherein the mucosal tissue is selected from the group of an oral mucosal tissue and a vaginal mucosal tissue; wherein the at least one active agent contained in the effervescent layer is released from the drug delivery device along with the at least one permeation enhancer within ten minutes, more preferably within five minutes, most preferably within one minute, of administration of the drug delivery device to the subject and wherein the at least one active agent contained in the mucoadhesive layer is released from the drug delivery device over a period of at least 8 hours, more preferably at least 12 hours, most preferably at least 24 hours, with near zero-order kinetics, more preferably with zero-order kinetics.

### BRIEF DESCRIPTION OF THE DRAWING

There are shown in the drawings certain exemplary embodiments of the present invention as presently preferred. It should be understood that the present invention is not limited to the embodiments disclosed as examples, and is capable of variation within the spirit and scope of the appended claims.

In the drawings,

FIG. 1 is a depiction of a preferred, two layer drug delivery device of the present invention,

FIG. 2 is depicts a graphical representation of the preferred release pattern for active agent(s) from the preferred drug delivery devices of the present invention; a graphical representation of the predicted plasma profile associated with such active agent(s) release pattern and the predicted therapeutic effect associated with such active agent(s) release patter;

FIG. 3 is a graphical representation of the mucoadhesiveness data collected for the drug delivery devices produced according to Examples A-D;

FIG. 4 is a graphical representation of the hardness data collected for the drug delivery devices produced according to Examples A-D; and,

FIG. 5 is a graphical representation of the release profiles for Granisetron measured from the drug delivery devices produced and tested according to Examples A-E.

### DETAILED DESCRIPTION

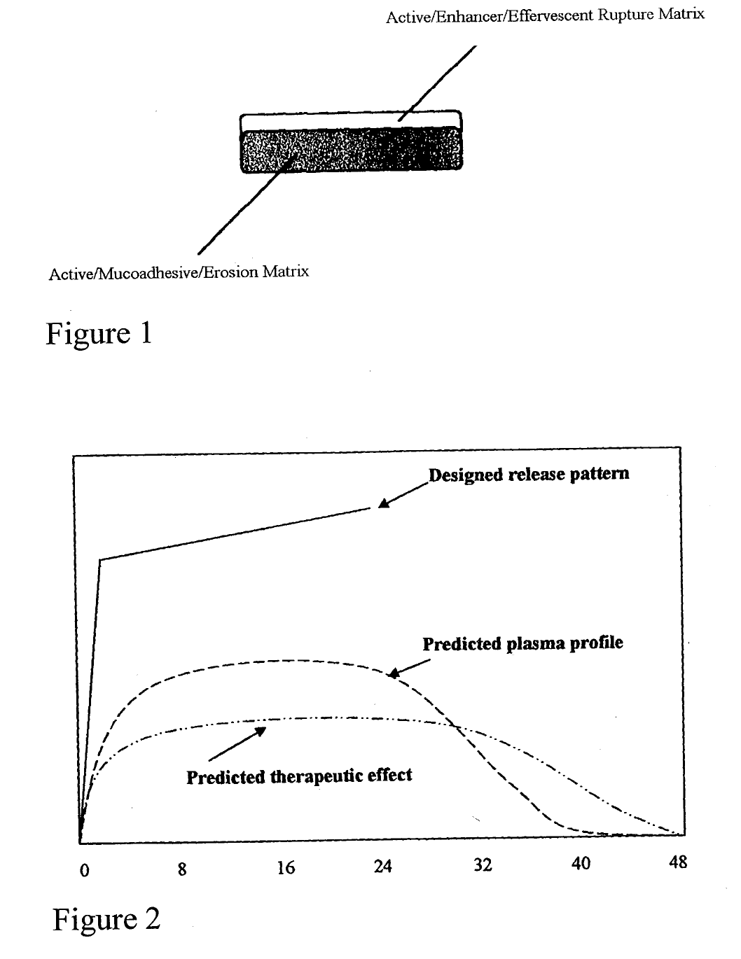

The drug delivery devices of the present invention provide a dual release system. An illustration of a preferred two layered drug delivery device of the present invention is depicted in FIG. 1. The active agent or combination of active agents included in the effervescent layer are rapidly released from the drug delivery device upon administration to a subject. The active agent or combination of active agents included in the mucoadhesive layer are released more slowly over an extended release period. Accordingly, the drug delivery devices of the present invention can preferably be designed to provide a rapid onset of therapeutic affect upon administration to a subject through the rapid release of active agent(s) from the effervescent layer. The drug delivery devices of the present invention can also preferably be designed to provide a controlled, sustained release of the active agent(s) from the mucoadhesive layer to provide continued therapeutic affect for extended periods, preferably at least 8 hours, more preferably at least 12 hours, most preferably at least 24 hours. In a most preferred configuration, the active agent(s) exhibit a near zero-order release from the mucoadhesive layer, more preferably zero-order release. The preferred release pattern for the drug delivery devices of the present invention is depicted in FIG. 2. Also depicted in FIG. 2 are the predicted plasma profile and predicted therapeutic effect determined using methods well known in the art based on the preferred release pattern.

The rate at which the active agent or combination of active agents included in the effervescent layer are released from the drug delivery device may be modified and controlled by a number of different factors. For example, the particular effervescent couple or combination of effervescent couples included in the effervescent layer, the concentration of the effervescent couple or combination of effervescent couples included in the effervescent layer and the dimensions of the drug delivery device may influence the rate at which the effervescent layer disintegrates upon administration of the drug delivery device to a subject.

The release rate at which the active agent or combination of active agents included in the mucoadhesive layer is determined by a number of different factors. For example, the dimensions of the mucoadhesive layer, the concentration of the active agent or combination of active agents, the solubility of the active agent or combination of active agents at the mucosal tissue to which the drug delivery device is adhered, how the active agent or combination of active agents is dispersed within the mucoadhesive layer and the density of the mucoadhesive layer may all influence the rate at which the active agent or combination of active agents is released from the device.

The thicknesses of a given layer in the drug delivery device is a factor in determining the rate of dissolution of that layer. A thick layer will dissolve more slowly than an otherwise similar thin layer. A thick layer may be desirable over a similar thin layer for larger dosages of active agent(s) based on the relative holding capacity of such layers.

The extent of the uptake of the active agent or combination of active agents from the drug delivery device at the site of application can be controlled by the dissolution rate of the drug delivery device. The drug delivery devices of the present invention release the active agent or combination of active agents contained therein as the drug delivery device dissolves or erodes. Once released from the drug delivery device, the active agent or combination of active agents may be absorbed by the mucosal tissue at or in proximity to the site of application or may be carried away to another location in the subject where it can be absorbed. For example, the active agent or combination of active agents may be released by the drug delivery device into the mouth of a subject after which much of the active agent or combination of active agents are subsequently swallowed and taken up in the gastrointestinal tract. In contrast, the active agent or active agents may be released by the drug delivery device into the mouth of a subject after which much active agent or combination of active agents are subsequently absorbed through the mucosal tissue in proximity to the drug delivery device.

A further parameter affecting the uptake of an active agent or combination of active agents from the drug delivery devices of the present invention is the manner in which the active agent or combination of active agents are dispersed in the drug delivery device. For example, the active agent or combination of active agents may be dispersed as colloidal particles or be microencapsulated within one or more of the layers of the drug delivery device or alternatively may be mixed throughout one or more of the layers of the drug delivery device as a reagent during casting such one or more layers.

The mucoadhesive layers of the drug delivery devices of the present invention may be formulated and designed to adhere to various mucosal tissue surfaces, preferably oral or vaginal mucosal tissue, most preferably buccal tissue. Depending on the design and purpose of a given drug delivery device, the site of adhesion may also be the site of administration for the active agent or combination of active agents incorporated into the drug delivery device. To provide stable bioadhesiveness over a broad range of both acidic and basic pH, nonionic water soluble polymers having hydroxyl functional groups are combined with anionic polymers to reduce the pH effect on the hydration, swelling and bioadhesion of the resulting polymer mix. The mixture of anionic and nonionic polymers is believed to result in hydrogen bonding which leads to a strong cross-linking force between the polymers. The ratio of anionic to nonionic polymers is a design feature which can be used to alter the release characteristics of the active agent or combination of active agents from a given mucoadhesive layer. It is also believed that the combination of nonionic polymers and anionic polymers can be selected such that the resulting polymeric matrix exhibits hydration, swelling, drug release and bioadhesion characteristics which are insensitive to the anticipated pH ranges to which the drug delivery devices will be exposed upon administration to the subject.

A swelling modifier or combination of swelling modifiers, which may include retarders and promoters, are preferably included in the mucoadhesive layer in the drug delivery devices of the present invention to further control the hydration rate, the degree of swelling and the active agent release kinetics. The anionic polymer(s), nonionic polymer(s) and swelling modifier(s) used in the mucoadhesive layer of the drug delivery devices of the present invention preferably should be homogeneously mixed at the molecular level through the aid of a suitable solvent.

A buffering agent or combination of buffering agents are preferably incorporated into the mucoadhesive layer of the drug delivery devices of the present invention to modulate the pH at the site of application and to reduce the incidence of mucosal irritation which might otherwise result from the acidity of the anionic polymers included in the mucoadhesive layer. The use of buffering agents in the mucoadhesive layer may also help facilitate a near zero-order release, more preferably a zero-order release, of the active agent or combination of active agents therefrom by modulating the pH at the site of application.

The drug delivery devices of the present invention preferably exhibit the following characteristics, namely (a) they should be sufficiently flexible to adapt to the opening exposing, and the surface of, the mucosal tissue to which they are adapted to be administered, (b) they should be comfortable and unobstructive during use, (c) they should be easy to administer to the site of application, (d) they should remain in place on the mucosal tissue without moving once administered thereto, (e) they should be capable of providing a burst of active agent or combination of active agents almost immediately subsequent to administration followed by a sustained, controlled release of the active agent or combination of active agents, preferably, a near zero-order release, most preferably a zero-order release, for an extended release period following administration, preferably at least 8 hours, more preferably at least 12 hours, most preferably at least 24 hours; (f) they should not cause irritation and (g) they should be completely dissolved and/or eroded at the end of the release period without the need for the physical removal of a residue.

The drug delivery devices of the present invention are intended to be inserted by the subject to be treated and do not require fitting by a physician. They can be easily inserted digitally or with an applicator.

In use, the drug delivery devices of the present invention are administered to a mucosal tissue of a subject and are maintained in intimate contact with the mucosal tissue for an extended release period, preferably for a release period of at least 8 hours, more preferably at least 12 hours, most preferably at least 24 hours, depending on the site of application, following in vivo placement of the drug delivery device. Upon contact with the moist mucous tissue of the subject, the drug delivery devices of the present invention provide a burst release of an active agent or combination of active agents from the effervescent layer followed by a sustained, controlled release of the active agent or combination of active agents from the mucoadhesive layer, as that layer naturally and slowly begins to dissolve and/or erode. As the mucoadhesive layer dissolves and/or erodes, it continually releases the active agent or combination of active agents at a rate which is sufficient to maintain a therapeutic effect, preferably at a rate which is near zero-order, most preferably at a rate which is zero order.

The active agent or combination of active agents included in the drug delivery devices of the present invention may be absorbed through the mucosal tissue at and in proximity to the site of administration of the drug delivery device, thereby avoiding any undesirable hepatic first-pass metabolism and gastrointestinal incompatibility associated with the active agent or combination of active agents. For example, one skilled in the art will recognize that a vaginal drug delivery device of the present invention can be used, for example, to administer an antifungal or other active agent for local treatment of, for example, vaginal infections such as yeast and bacterial infections. It will be recognized, however, that the active agent or combination of active agents can also be absorbed into the body at a location not in proximity to the site of administration of the drug delivery device. For example, one skilled in the art will recognize that a buccal drug delivery device of the present invention can be used, for instance, to administer a active agent or combination of active agents which are absorbed into the body through the gastrointestinal system.

The drug delivery devices of the present invention preferably exhibit good adhesion to the mucosal tissue of the subject to which they are intended to be administered. Upon contact with the mucus which is excreted by the mucosal tissue, the drug delivery device preferably hydrates and adheres to the mucosal tissue. This feature permits the drug delivery devices of the present invention to be worn comfortably by the subject and to maintain the drug delivery device in a proper position to facilitate the sustained, controlled delivery of an active agent or combination of active agents to the subject.

The drug delivery devices of the present invention preferably contain: (a) at least one mucoadhesive layer, containing: (i) at least one nonionic polymer, (ii) at least one anionic polymer, (iii) at least one swelling modifier, (iv) at least one buffering agent; (b) at least one effervescent layer, containing: (i) at least one permeation enhancer, (ii) at least one effervescent couple, with each such effervescent couple containing an anhydrous acid and an alkalizing agent, and (iii) at least one binder; and (c) at least one active agent; wherein the at least one active agent is contained in both the at least one mucoadhesive layer and the at least one effervescent layer; wherein the at least one active agent contained in the at least one effervescent layer is released from the drug delivery device along with the at least one permeation enhancer within 10 minutes, more preferably within 5 minutes, most preferably within 1 minute, of administration of the drug delivery device to a subject through application of the drug delivery device to a mucosal tissue, preferably an oral mucosal tissue or a vaginal mucosal tissue and wherein the at least one active agent contained in the at least one mucoadhesive layer is released from the drug delivery device over a period of at least 8 hours, more preferably at least 12 hours, most preferably at least 24 hours, with near zero-order kinetics, more preferably with zero-order kinetics.

The drug delivery devices of the present invention may be used as a vehicle for delivering a wide range of active agents to a subject. For example, the active agent may include small molecules (i.e., less than 500 daltons), proteins, nucleic acids including antisense molecules or other biological or synthetic molecules. Active agents suitable for use with the present invention include, but are by no means limited to, therapeutic agents, nutritional supplements and hygiene aids.

Therapeutic agents suitable for use with the present invention include, but are not limited to, analgesics, -adrenergic receptor blockers, anti-Alzheimer's disease medication, antianginal, antianxiety, antiarrythmics, antiarthritics, antibiotics, anticoagulants/thrombolytics, anticonvulsants/anti-Parkinson medication, anti-depressants, anti-diabetics, anti-diarrheal, anti-epilectics, anti-fungal, anti-gout, anti-heartworm medication for dogs, anti-histamines, anti-hypertensives, anti-inflammatories, anti-infectives, antimigraines, anti-nasuants/anti-emetics, anti-neoplastics/anti-tumor drugs, anti-pruitics, anti-psychotics, anti-pyretics, anti-spasmodics, anti-virals, bronchial dilators/anti-asthmatics, calcium antagonists, cardiac agents, cardiotonics, central nervous system actives, contraceptives, coronary vasodilators, cough/cold remedies, dietary supplements, including vitamins and minerals, diuretics, fertility drugs, flea control agents for animals (Ivermectin), H₂ receptor antagonists, herbal actives, hormones, hypoglycemics, hypolipidermcs, muscle relaxants, ovulation stimulators, peptide drugs, polypeptide drugs, proteins such as insulin, calcitonin, LHRH, sedatives and hypnotics, sexual dysfunction drugs, sleep aids, smoking cessation aids, steroids and steroidals, tranquilizers, laxatives, ophthalmic preparations, breath fresheners, breath deodorants, saliva substitutes, antigingivitis agents, anti-cavity agents, anti-plaque agents, diagnostic indicators, and local anesthetics. Also included are drugs for treatment of osteoporosis, hormone replacement, treatment of periodontal disease, antiseptics, corticosteroids, non steroidal anti-inflammatory agents, anti-viral agents and vaccines. Preferably, the bioadhesive, closed-cell foam film, sustained release, delivery devices of the present invention contain 0.01 to 50 wt % of an active agent or combination of active agents. Preferably, the mass of the active agent or combination of active agents contained in the drug delivery devices of the present invention is divided up between the mucoadhesive layer and the effervescent layer at a ratio of 9:1 to 1:1.

Nonionic polymers suitable for use with the present invention include, but are by no means limited to, cellulose derivatives such as carboxymethylcellulose, hydroxyethyl cellulose, methylcellulose, hydroxypropyl cellulose and hydroxypropyl methylcellulose; polyvinylpyrrolidone; polyvinyl alcohol; polyethylene oxide; modified starch; gelatin; agar; guar gum; locust bean gum; bentonite and scheroglucan; preferably polyvinylpyrrolidone, hydroxypropyl methylcellulose, hydroxypropyl cellulose, polyvinyl alcohol, gelatin, polyethylene oxide; most preferably, polyvinyl alcohol, gelatin and hydroxypropyl methylcellulose. The nonionic polymer or combination of nonionic polymers included in the drug delivery devices of the present invention preferably exhibit an equilibrium moisture content of 10 to 20 wt % measured at 20° C. and 75% relative humidity. Preferably, the drug delivery devices of the present invention contain 10 to 60 wt % of a nonionic polymer or combination of nonionic polymers.

Anionic polymers suitable for use with the present invention include, but are by no means limited to, polyacrylic acid such as carbopol, polycarbophil, poly(methyl vinyl ether-co-methacrylic acid), poly(2-hydroxyethyl methacrylate), poly(methylmethacrylate), poly(isobutylcyanoacrylate), poly(isohexycyanoacrylate) and polydimethylaminoethyl methacrylate; acacia; alginate; carrageenan; guar gum derivative; karaya gum; pectin; tragacanth gum; xanthan gum; dextran; sodium carboxymethylcellulose (“sodium CMC”) and hyaluronic acid; preferably carbopol, polycarbophil, alginate, carrageenan, pectin and sodium CMC; most preferably carbopol, polycarbophil, alginate, carrageenan and sodium CMC. The anionic polymer or combination of anionic polymers included in the drug delivery devices of the present invention preferably exhibit a viscosity of at least 1,000 mPa*s measured at 20° C. for a 1% w/v aqueous solution. Preferably, the drug delivery devices of the present invention contain 10 to 60 wt % of an anionic polymer or combination of nonionic polymers.

Swelling modifiers suitable for use with the present invention may include either, or both, retarders and promoters. The drug delivery devices of the present invention preferably contain swelling modifiers in the mucoadhesive layer, with the swelling modifier or combination of swelling modifiers constituting 0.1 to 50 wt % of the mucoadhesive layer.

Retarders suitable for use with the present invention include, but are by no means limited to, wax such as beeswax, carnauba wax, paraffin wax, castorwax, spermaceti, petrolite and microcrystalline wax; hydrogenated oils and fats such as castor oil, theobroma oil, partially hydrogenated soybean oil, glyceryl laurates, glyceryl myristates, glyceryl palmitates and glyceryl stearates; long chain fatty acids and alcohols such as lauric acid/alcohol, myristic acid/alcohol, palmitic acid/alcohol, stearic acid/alcohol and oleic acid; ethylene glycol distearate; polyoxyethylene sorbitol beeswax derivative; sorbitan esters such as sorbitan tristearate, sorbitan sesquioleate and sorbitan monooleate; propylene glycol monostearate; lecithin and poloxamers; preferably polyoxyethylene glyceride fatty acid derivatives, sorbitan esters, lecithin, poloxamers, wax, hydrogenated vegetable oils. Preferably the retarder or combination of retarders included in the drug delivery devices of the present invention are lipophilic. Most preferably, the retarder or combination of retarders exhibit a hydrophilicity lipophilicity balance (“HLB”) of 5 or less.

Promoters suitable for use with the present invention include, but are by no means limited to, inorganic electrolytes such as sodium carbonate, sodium bicarbonate, potassium carbonate, potassium bicarbonate, sodium phosphate, potassium phosphate, triethanolamine, aminomethyl propanol, trimethylamine, tetrahydroxypropyl ethylenediamine, sodium chloride, magnesium chloride, ferric chloride and aluminum sulfate; and, sugars such as mannitol, sorbitol, lactose, xylitol, sucrose, sugar and dextrose; preferably sodium bicarbonate, potassium bicarbonate, sodium phosphate, potassium phosphate, triethanolamine, sodium chloride; most preferably sodium bicarbonate, potassium bicarbonate and triethanolamine.

Permeation enhancers suitable for use with the present invention include, but are by no means limited to, cationic surfactants such as cetrimide, cetrimonium bromide, benzalkonium chloride and cetylpyridinium chloride; anionic surfactants such as aluminum nonostearate, sodium lauryl sulfate, triethanolamine lauryl sulfate, sodium laurate and sodium docusate; nonionic surfactants such as glycol esters, glycerol esters, sorbitan derivatives, polyoxyethylene esters, polyoxyethylene ethers, poloxamers, nonylphenyl ethers and propylene glycol diacetate; steroidal surfactants such as sodium glycodeoxycholate, sodium taurodeoxycholate, sodium taurocholate, sodium cholate, sodium glycocholate, azone and sodium fusidate; fatty acids and derivatives such as sodium myristate, sodium laurate, oleic acid caprylic acid and palmitoylacrnitine; chelators such as EDTA and its salts, sodium salicylate, sodium citrate, DTPA and its salts, HEDTA and its salts and NTA; positively charged polymers such as chitosan, trimethyl chistosan and polyarginine; and, cyclodextrins. The drug delivery devices of the present invention preferably contain a permeation enhancer or a combination of permeation enhancers in the effervescent layer, with the permeation enhancer or combination of permeation enhancers constituting 0.01 to 20 wt % of the effervescent layer.

Effervescent couples suitable for use with the present invention include, but are by no means limited to, an acid component such as citric acid, tartaric acid, amalic acid, fumeric acid adipic acids and succinic acids; and a basic component such as alkaline carbonates and bicarbonates, for example sodium bicarbonate, sodium carbonate, potassium bicarbonate and magnesium carbonate. The drug delivery devices of the present invention preferably contain at least one effervescent couple in the effervescent layer, with the at least one effervescent couple constituting 50 to 95 wt % of the effervescent layer.

The drug delivery devices of the present invention may also optionally contain 0.01 to 10 wt % processing aids and taste modifiers in either, or both, of the mucoadhesive layer and the effervescent layer. Processing aids and taste masking agents suitable for use with the present invention include taste modifiers, coloring agents, glidants and lubricants.

Taste modifiers suitable for use with the present invention include flavoring agents, sweetening agents and taste masking agents. Preferred taste modifying agents include the essential oils or water soluble extracts of menthol, wintergreen, peppermint, sweet mint, spearmint, vanillin, cherry, chocolate, cinnamon, clove, lemon, orange, raspberry, rose, spice, violet, herbal, fruit, strawberry, grape, pineapple, peach, kiwi, papaya, mango, coconut, apple, coffee, plum, watermelon, nuts, durean, green tea, grapefruit, banana, butter, chamomile, sugar, dextrose, lactose, mannitol, sucrose, xylitol, malitol, acesulfame potassium, talin, glycyrrhizin, sucralose, aspartame, saccharin, sodium saccharin, sodium cyclamate and honey.

Coloring agents suitable for use with the present invention include, but are by no means limited to, FD & C coloring agents, natural coloring agents, natural juice concentrates and pigments. Preferred pigments include titanium oxide, silicon dioxide and zinc oxide. Glidants suitable for use with the present invention include, but are by no means limited to, magnesium stearate and talc. Glidants may be added to either one or both of the mucoadhesive layer and the effervescent layer, wherein the amount of glidants included in a given layer preferably ranges from 0.01 to 10 wt % of such layer. The addition of a glidant or combination of gliadants to a given layer may operate to improve the flowability during compression of the mixture of materials constituting that layer.

Lubricants suitable for use with the present invention include, but are by no means limited to, calcium stearate, glyceryl monostearate, glyceryl palmitostearate, hydrogenated castor oil, hydrogenated vegetable oil, light mineral oil, magnesium stearate, mineral oil, polyethylene glycol, sodium benzoate, sodium lauryl sulfate, sodium stearyl fumarate, stearic acid, talc and zinc stearate.

In a preferred embodiment of the present invention, drug delivery devices are provided which contain: (a) a mucoadhesive layer, containing: (i) 10 to 60 wt % nonionic polymer or combination of nonionic polymers, (ii) 10 to 60 wt % anionic polymer or combination of anionic polymers, (iii) 0.1 to 50 wt % swelling modifier or combination of swelling modifiers, (iv) 0.1 to 10 wt % buffering agent or combination of buffering agents; (b) an effervescent layer, containing: (i) 0.01 to 20 wt % permeation enhancer or combination of permeation enhancers, (ii) 50 to 95 wt % effervescent couple or combination of effervescent couples, each such effervescent couple including an anhydrous acid and an alkalizing agent, and (iii) 0.01 to 10 wt % binder or combination of binders; (c) 0.01 to 50 wt % active agent or combination of active agents; (d) 0.01 to 10 wt % glidant or combination of glidants. Preferably the active agent or combination of active agents is contained in both the mucoadhesive layer and the effervescent layer. Preferably, the glidant or combination of glidants is contained in both the mucoadhesive layer and the effervescent layer. Preferably, the mass ratio of anionic to non-ionic polymers in the mucoadhesive layer is 1:6 to 6:1. Preferably, the mass ratio of the swelling modifier(s) to the combined mass of the anionic polymer(s) and the nonionic polymer(s) is 1:50 to 1:1. Preferably, the drug delivery devices of the present invention completely erode from the site of application on the mucosal tissue and do not leave behind a residue which must be removed from the cite of application. Preferably, the active agent or combination of active agents contained in the effervescent layer is released from the drug delivery device along with the permeation enhancer or combination of permeation enhancers within 10 minutes, more preferably within 5 minutes, most preferably within 1 minute, of administration of the drug delivery device to a subject through application of the drug delivery device to a mucosal tissue, preferably an oral mucosal tissue or a vaginal mucosal tissue. Preferably, the active agent or combination of active agents contained in the mucoadhesive layer is released from the drug delivery device over a period of at least 8 hours, more preferably at least 12 hours, most preferably at least 24 hours, with near zero-order kinetics, more preferably with zero-order kinetics.

One skilled in the art given the above description of the drug delivery devices of the present invention will be able to produce those devices using a variety of known processing methods. Preferably, the drug delivery devices of the present invention are produced using the following described process.

A preferred method for preparing the drug delivery devices of the present invention involves:

I. Blending the components of the mucoadhesive layer, namely:

(a) the nonionic polymer or combination of nonionic polymers,

(b) the anionic polymer or combination of anionic polymers,

(c) the swelling modifier or combination of swelling modifiers,

(d) the buffering agent or combination of buffering agents,

(e) the active agent or combination of active agents, and

(f) any additional optional additives;

II. Blending the components of the effervescent layer, namely:

(a) the anhydrous acid or combination of anhydrous acids and the alkalizing agent or combination of alkalizing agents making up the effervescent couple or combination of effervescent couples,

(b) the binder or combination of binders,

(c) the active agent or combination of active agents, and

(d) any additional optional additives,

III. Pressing the blended components of the mucoadhesive layer into a layer; and,

IV. Pressing the blended components of the effervescent layer into a layer on top of the mucoadhesive layer produced in III.

The drug delivery devices of the present invention may be administered to a subject by placing the exposed surface of the mucoadhesive layer on a mucosal tissue of the subject. Upon application to a mucosal tissue, the drug delivery device will adhere to the mucosal tissue and the effervescent layer will quickly dissolve or erode, preferably within 10 minutes, more preferably within 5 minutes, providing a bursting release of active agent or combination of active agents to the subject. The mucoadhesive layer of the drug delivery device will dissolve or erode in a controlled fashion over an extended period of time providing a controlled release of active agent or combination of active agents to the subject. Preferably, the mucoadhesive layer will continue to release the active agent or active agents for a period of at least 8 hours, more preferably at least 12 hours, most preferably at least 24 hours. The release of the active agent or active agents from the mucoadhesive layer preferably follows near zero-order kinetics, most preferably, zero-order kinetics.

### EXAMPLES

The preferred embodiments of the present invention will now be further described through the following examples set forth hereinbelow which are intended to be illustrative of the preferred embodiments of the present invention and are not intended to limit the scope of the invention as set forth in the appended claims.

### Example A

A drug delivery device of the present invention was prepared as follows:

(a) prepared a mucoadhesive controlled powder mix by mixing:

(i) 47.5 g of hydroxypropylmethylcellulose (Methocel E5 commercially available from Dow Chemical Company),

(ii) 47.4 g of carboxyvinyl polymer (Carbopol 971 commercially available from BF Goodrich),

(iii) 2 g glycerol dibehenate Ep-glyceryl behenate (Compritol commercially available from Gattefossé),

(iv) 0.8 g sodium bicarbonate, and

(v) 2.4 gram Granisetron base powder;

(b) prepared a bursting release powder mix by mixing

(i) 1.8 g sodium EDTA,

(ii) 4.5 g Granisetron base powder,

(iii) 2.7 g hydroxypropylmethylcellulose (Methocel E5),

(iv) 0.9 g magnesium stearate,

(v) 36.04 g anhydrous citric acid, and

(vi) 54.05 g sodium bicarbonate

(c) compressed 83 mg of the mocoadhesive controlled powder mix to form a mucoadhesive layer;

(d) compressed 22 mg of bursting release powder mix onto the top of the mucoadhesive layer forming a bursting release layer (effervescent layer) thereon to provide a bilayer drug delivery device.

### Example B

Another drug delivery device of the present invention was prepared as follows:

(a) prepared a mucoadhesive controlled powder mix by mixing:

(i) 28.5 g of hydroxypropylmethylcellulose (Methocel E5 commercially available from Dow Chemical Company),

(ii) 28.5 g of carboxyvinyl polymer (Carbopol 971 commercially available from BF Goodrich),

(iii) 38.7 g mannitol,

(iv) 1.2 g glycerol dibehenate Ep-glyceryl behenate (Compritol commercially available from Gattefossé),

(iv) 0.8 g sodium bicarbonate, and

(v) 2.4 gram Granisetron base powder;

(b) prepared a bursting release powder mix by mixing

(i) 1.8 g sodium EDTA,

(ii) 4.5 g Granisetron base powder,

(iii) 2.7 g hydroxypropylmethylcellulose (Methocel E5),

(iv) 0.9 g magnesium stearate,

(v) 36.04 g anhydrous citric acid, and

(vi) 54.05 g sodium bicarbonate

(c) compressed 83 mg of the mocoadhesive controlled powder mix to form a mucoadhesive layer;

(d) compressed 22 mg of bursting release powder mix onto the top of the mucoadhesive layer forming a bursting release layer (effervescent layer) thereon to provide a bilayer drug delivery device.

### Example C

Another drug delivery device of the present invention was prepared as follows:

(a) prepared a mucoadhesive controlled powder mix by mixing:

(i) 44.5 g of hydroxypropylmethylcellulose (Methocel E5 commercially available from Dow Chemical Company),

(ii) 44.5 g of carboxyvinyl polymer (Carbopol 971 commercially available from BF Goodrich),

(iii) 7.8 g glycerol dibehenate Ep-glyceryl behenate (Compritol commercially available from Gattefossé),

(iv) 0.8 g sodium bicarbonate, and

(v) 2.4 gram Granisetron base powder;

(b) prepared a bursting release powder mix by mixing

(i) 1.8 g sodium EDTA,

(ii) 4.5 g Granisetron base powder,

(iii) 2.7 g hydroxypropylmethylcellulose (Methocel E5),

(iv) 0.9 g magnesium stearate,

(v) 36.04 g anhydrous citric acid, and

(vi) 54.05 g sodium bicarbonate

(c) compressed 83 mg of the mocoadhesive controlled powder mix to form a mucoadhesive layer;

(d) compressed 22 mg of bursting release powder mix onto the top of the mucoadhesive layer forming a bursting release layer (effervescent layer) thereon to provide a bilayer drug delivery device.

### Example D

Another drug delivery device of the present invention was prepared as follows:

(a) prepared a mucoadhesive controlled powder mix by mixing:

(i) 26.8 g of hydroxypropylmethylcellulose (Methocel E5 commercially available from Dow Chemical Company),

(ii) 26.8 g of carboxyvinyl polymer (Carbopol 971 commercially available from BF Goodrich),

(iii) 38.7 g mannitol,

(iv) 4.6 g glycerol dibehenate Ep-glyceryl behenate (Compritol commercially available from Gattefossé),

(iv) 0.8 g sodium bicarbonate, and

(v) 2.4 gram Granisetron base powder;

(b) prepared a bursting release powder mix by mixing

(i) 1.8 g sodium EDTA,

(ii) 4.5 g Granisetron base powder,

(iii) 2.7 g hydroxypropylmethylcellulose (Methocel E5),

(iv) 0.9 g magnesium stearate,

(v) 36.04 g anhydrous citric acid, and

(vi) 54.05 g sodium bicarbonate

(c) compressed 83 mg of the mocoadhesive controlled powder mix to form a mucoadhesive layer;

(d) compressed 22 mg of bursting release powder mix onto the top of the mucoadhesive layer forming a bursting release layer (effervescent layer) thereon to provide a bilayer drug delivery device.

### Example E

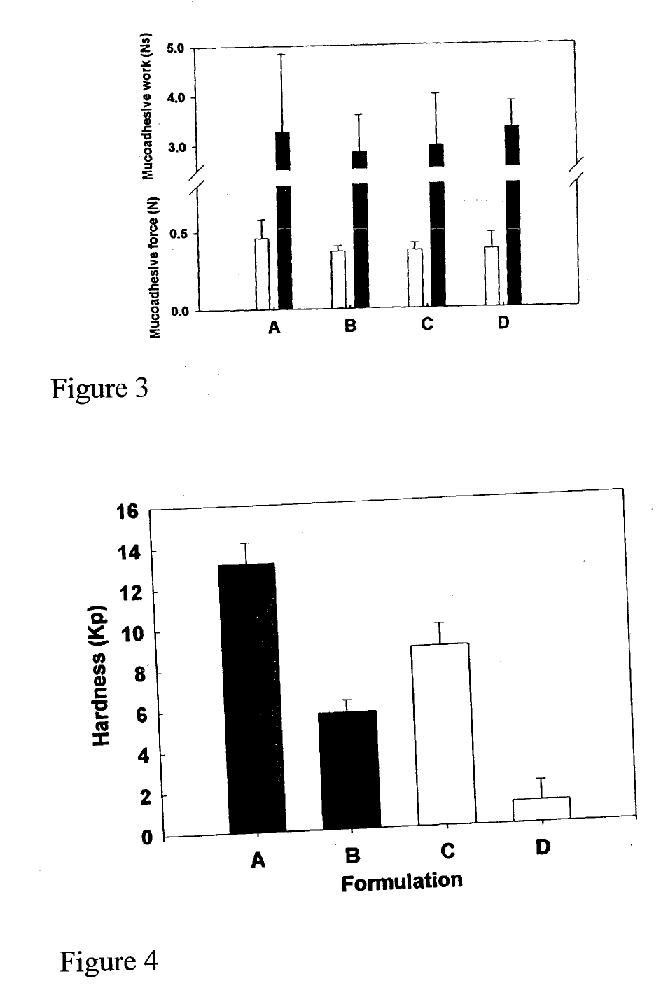

The drug delivery devices produced according to Examples A-D were tested for mucoadhesive force and hardness. The results of these analyses are presented in graphical form in FIGS. 3 & 4, respectively.

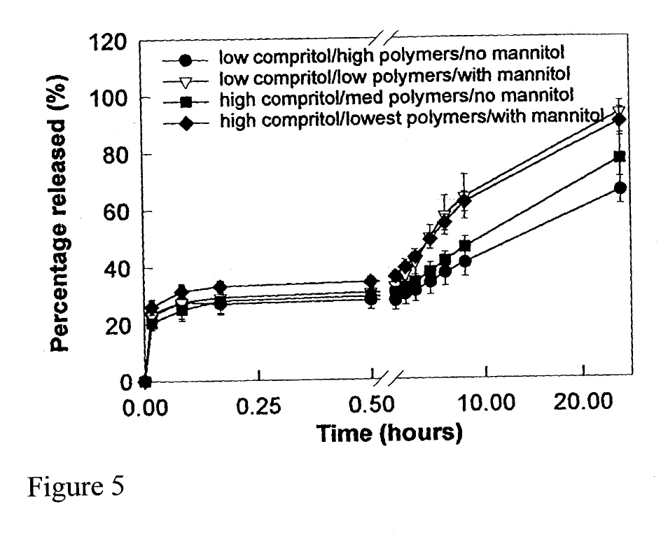

The release profiles of the drug delivery devices produced according the Examples A-D for Granisetron were generated using dissolution data obtained using USP apparatus I. Specifically, the mucoadhesive layer of the subject drug delivery devices was taped down on a wire plate and a wire mesh was used to cover the bursting release layer (effervescent layer). The devices were then contacted with a dissolution medium (pH 7 phosphate buffer solution) which was maintained at 37° C. The rotation speed in the apparatus was 50. Samples were extracted at each of the predetermined time intervals, namely 1, 5, 10, 30 minutes, 1, 2, 3, 4.5, 6, 8 and 24 hours. The samples were filtered and analyzed by HPLC. The results of this analysis for Granisetron are presented in graphical form in FIG. 5.

The present invention having been disclosed in connection with the foregoing embodiments, additional embodiments will now be apparent to persons skilled in the art. The present invention is not intended to be limited to the embodiments specifically mentioned, and accordingly reference should be made to the appended claims rather than the foregoing discussion, to assess the spirit and scope of the present invention in which exclusive rights are claimed.

We claim:

1. A drug delivery device, comprising:
(a) a mucoadhesive layer, comprising:
(i) at least one non-ionic polymer,
(ii) at least one anionic polymer,
(iii) at least one swelling modifier, and
(iv) at least one buffering agent;
(b) an effervescent layer, comprising:
(i) at least one permeation enhancer,
(ii) an effervescent couple, comprising an anhydrous acid and an alkalizing agent, and
(iii) at least one binder; and,
(c) at least one active agent;
wherein the at least one active agent is contained in both the mucoadhesive layer and the effervescent layer; wherein the at least one active agent contained in the effervescent layer is released from the drug delivery device along with the at least one permeation enhancer within 10 minutes of administration of the drug delivery device to a subject through application of the drug delivery device to a mucosal tissue selected from the group consisting of an oral mucosal tissue and a vaginal mucosal tissue and wherein the at least one active agent contained in the mucoadhesive layer is released from the drug delivery device over a period of at least 8 hours with near zero-order kinetics.

2. The drug delivery device of claim 1, wherein the at least one active agent contained in the effervescent layer is released from the drug delivery device within five minutes of administration of the drug delivery device.

3. The drug delivery device of claim 1, wherein the at least one active agent contained in the effervescent layer is released from the drug delivery device within one minute of administration of the drug delivery device.

4. The drug delivery device of claim 1, wherein the at least one active agent is released from the drug delivery device over a period of at least 12 hours with near zero-order kinetics.

5. The drug delivery device of claim 1, wherein the at least one active agent is released from the drug delivery device over a period of at least 24 hours with near zero-order kinetics.

6. The drug delivery device of claim 1, wherein the effervescent layer is nonmucoadhesive.

7. The drug delivery device of claim 1, wherein the mass ratio of anionic to nonionic polymers in the mucoadhesive layer is 1:6 to 6:1.

8. The drug delivery device of claim 1, wherein the mass ratio of (a) the at least one swelling modifier to (b) the combined mass of the at least one anionic polymer and the at least one non-ionic polymer is 1:50 to 1:1.

9. The drug delivery device of claim 1, wherein the drug delivery device completely erodes from the site of application on the mucosal tissue.

10. The drug delivery device of claim 1, wherein the at least one swelling modifier is selected from the group consisting of swelling retarders and swelling promoters.

11. The drug deliver device of claim 1, further comprising:
(d) at least one glidant;
wherein the at least one glidant is present in both the mucoadhesive layer and the effervescent layer.

12. The drug delivery device of claim 1, wherein the at least one active agent comprises 0.01 to 50 wt % of the mucoadhesive layer.

13. The drug delivery device of claim 1, wherein at least one non-ionic polymer comprises 10 to 60 wt % of the mucoadhesive layer.

14. The drug delivery device of claim 1, wherein the at least one anionic polymer comprises 10 to 60 wt % of the mucoadhesive layer.

15. The drug delivery device of claim 1, wherein the at least one swelling modifier comprises 0.1 to 50 wt % of the mucoadhesive layer.

16. The drug delivery device of claim 1, wherein at least one permeation enhancer comprises 0.01 to 20 wt % of the effervescent layer.

17. The drug delivery device of claim 1, wherein the at least one effervescent couple comprises 50 to 95 wt % of the effervescent layer.

18. The drug delivery device of claim 1, further comprising one or more additional ingredients selected from the group consisting of: taste modifiers, coloring agents, glidants and lubricants.

19. The drug delivery device of claim 18, wherein the one or more additional ingredients comprise 0.01 to 10 wt % of the drug delivery device.

20. A drug delivery device, comprising:
(a) a mucoadhesive layer, comprising:
(i) at least one non-ionic polymer,
(ii) at least one anionic polymer,
(iii) at least one swelling modifier, and
(iv) at least one buffering agent;
(b) an effervescent layer, comprising:
(i) at least one permeation enhancer,
(ii) an effervescent couple, comprising an anhydrous acid and an alkalizing agent, and
(iii) at least one binder;
(c) at least one active agent; and,
(d) at least one glidant;
wherein the at least one active agent is contained in both the mucoadhesive layer and the effervescent layer; wherein the at least one glidant is present in both the mucoadhesive layer and the effervescent layer; wherein the mass ratio of anionic to non-ionic polymers in the mucoadhesive layer is 1:6 to 6:1; wherein the mass ratio of the (I) at least one swelling modifier to the (II) combined mass of the at least one anionic polymer and the at least one non-ionic polymer is 1:50 to 1:1; wherein the drug delivery device completely erodes from the site of application on the mucosal tissue; wherein the at least one active agent contained in the effervescent layer is released from the drug delivery device along with the at least one permeation enhancer within 10 minutes of administration of the drug delivery device to a subject through application of the drug delivery device to a mucosal tissue selected from the group consisting of an oral mucosal tissue and a vaginal mucosal tissue and wherein the at least one active agent contained in the mucoadhesive layer is released from the drug delivery device over a period of at least 8 hours with near zero-order kinetics.

21. A method for delivery active agents to a subject, comprising the administration of a drug delivery device to a mucosal tissue of the subject, wherein the drug delivery device comprises:
(a) a mucoadhesive layer, comprising:
(i) at least one non-ionic polymer,
(ii) at least one anionic polymer,
(iii) at least one swelling modifier, and
(iv) at least one buffering agent;
(b) an effervescent layer, comprising:
(i) at least one permeation enhancer,
(ii) an effervescent couple, comprising an anhydrous acid and an alkalizing agent, and
(iii) at least one binder; and,
(c) at least one active agent;
wherein the at least one active agent is contained in both the mucoadhesive layer and the effervescent layer; wherein the mucosal tissue is selected from the group consisting of an oral mucosal tissue and a vaginal mucosal tissue; wherein the at least one active agent contained in the effervescent layer is released from the drug delivery device along with the at least one permeation enhancer within 10 minutes of administration of the drug delivery device to the subject and wherein the at least one active agent contained in the mucoadhesive layer is released from the drug delivery device over a period of at least 8 hours with near zero-order kinetics.
